(19) United States
(12) Patent Application Publication
(10) Pub. No.: US 2016/0228640 A1
(43) Pub. Date: Aug. 11, 2016

(54) METHOD AND SYSTEM FOR INTERACTING WITH AN ENVIRONMENT

(71) Applicant: MC10, Inc., Lexington, MA (US)

(72) Inventors: Jesus PINDADO, Lexington, MA (US); Shyamal PATEL, Somerville, MA (US); Milan RAJ, Cambridge, MA (US); Roozbeh GHAFFARI, Cambridge, MA (US); Pinghung WEI, Burlingame, CA (US); Nirav SHETH, Bedford, MA (US)

(21) Appl. No.: 15/016,937

(22) Filed: Feb. 5, 2016

Related U.S. Application Data
(60) Provisional application No. 62/117,022, filed on Feb. 17, 2015.
(60) Provisional application No. 62/112,242, filed on Feb. 5, 2015.

(51) Int. Cl. A61M 5/172; A61N 1/36; A61N 1/365; A61B 5/0402; A61B 5/0488; A61B 5/0476; A61B 5/145; A61B 5/00; A61B 5/01; A61B 5/08; A61B 5/024; A63F 13/212; G05D 23/19; G01J 1/42; G06F 17/30

(52) U.S. Cl. CPC A61M 5/1723; G06F 17/30386; A61N 1/36139; A61N 1/36514; A61B 5/0402; A61B 5/0488; A61B 5/0476; A61B 5/14517; A61B 5/14532; A61B 5/14546; A61B 5/14542; A61B 5/442; A61B 5/01; A61B 5/0816; A61B 5/024; A63F 13/212; G05D 23/1917; G01J 1/42; A61B 2560/0242

(57) ABSTRACT

A system for controlling environmental parameters can include one or more body worn sensor devices that detect and report one or more physical, physiological, or biological parameters of a person in an environment. The sensor devices can communicate sensor data indicative of the one or more physical, physiological, or biological parameters of a person to a hub that processes the data and communicates with one or more devices or systems that can be used to change environmental. In some embodiments, the environment includes a device or machine, such as a motorized vehicle and the hub can communicate with the device or machine to cause a change in the operation or function of the device or machine. For example, the motorized vehicle can be caused to stop or slow down in response to sensor data indicating that the operator is experiencing stress or becomes disabled.

### CROSS-REFERENCE TO RELATED APPLICATIONS

This application claims benefit under 35 U.S.C. §119(e) of the U.S. Provisional Application No. 62/112242, filed Feb. 5, 2015 and No. 62/117022, filed Feb. 17, 2015, the contents of both of which are incorporated herein by reference in their entirety.

### STATEMENT REGARDING FEDERALLY SPONSORED RESEARCH

Not Applicable

### REFERENCE TO MICROFICHE APPENDIX

Not Applicable

### BACKGROUND

1. Technical Field of the Invention

The present invention is directed to methods and systems for monitoring physical, physiologic and/or biologic information about one or more persons or subjects and using physical, physiologic and/or biologic information or information derived from physical, physiologic and/or biologic information, to interact with environmental systems and devices. More specifically, the system can include one or more sensors that detect a condition of one or more persons or subjects and use that information to change the operation of the system or a device in communication with the system.

2. Description of the Prior Art

Many of the existing health and wellness aides utilize sensors to detect and record various physical and physiologic information about a person or a subject. These devices use sensors, such as accelerometers, to measure movement and store this information for later presentation in various forms to the person wearing the device or other person, such as their coach. These devices assist in training by enabling the user to review historic information and use this information to make subsequent decisions.

Other systems utilize body worn or carried devices to provide some form of convenient environmental control. The systems, typically referred to as Home Automation, utilize information available from a smart phone or similar device to automate some control of the home environment. For example, some thermostats include sensors that detect the presence or absence of people in a room or zone and attempt to reduce energy costs by automatically reducing the heating or cooling of the zone until activity is detected. Other thermostat systems can use the location information available from a smart phone to determine when and how to control the heating and cooling of a home. When, the system detects that the person is away from home, the thermostat automatically turns the heating or cooling system down or off until the person moves within a predefined distance to the home, at which point the thermostat turns the heating or cooling system on to make the home comfortable for the person's arrival.

### SUMMARY

The present invention is direct to systems that are adapted to modify the operation or condition of one or more environments (or systems that influence the state of one or more environments) or systems that control the operation of a machine, as function of one or more sensed conditions of one or more people or subjects (which includes inanimate objects). The present invention is directed to methods for monitoring physical, physiologic and/or biologic conditions of a person or subject and using this information, either alone or in combination with other information, to influence or control, either directly or indirectly, one or more environmental factors or the operation of a system or machine.

In accordance with the invention, one or more people and/or subjects can be monitored by one or more sensing devices that indicate one or more conditions of some or all of the people and/or subjects. The conditions can include physical conditions, such as location and motion of the person or subject or a part of the person or subject. The conditions can include physiologic or biologic conditions, such as the mechanical, physical, thermal and/or biochemical aspects of functions and/or processes of the person or subject. The conditions can include mental, emotional, and psychiatric conditions, such as, mood, focus, concentration, depression, and alertness.

The sensed information about one or more persons or subjects can be collected and processed or analyzed and used as an input or used to select or modify an input to a control system that controls the person or subject's environment, or a machine or device related to the person or subject.

The system can utilize one or more algorithms to determine whether to modify the environment or the operation of a system or machine. For example, the algorithm can compare one or more parameters representative of one or more sensed conditions to a predefined threshold value (or range) and based on the outcome of the comparison, take no further action or proceed to interact with a control system to cause a change in an environment or the operation of a machine.

In accordance with some embodiments, the system, according to the algorithm, can include additional data as inputs to determine whether to interact with the control system to cause a change in an environment or the operation of a machine. The additional data can be data obtained from local and remote sources, such as environmental data (e.g., temperature, barometric pressure, humidity, wind velocity and wind direction), time of day, ambient noise levels (e.g., levels of music playing or background noise), and ambient light levels (e.g., time of day, whether lights are on, whether is sunny or cloudy outside). The system can process these data values using a logic tree or a set of rules to determine whether to interact with the control system to cause a change in an environment or the operation of a machine.

In accordance with some embodiments of the invention, the system, according to the algorithm, can determine a trend or a rate of change of one or more parameters and use the rate of change to predict an event time in the future when a specific parameter could exceed a threshold and require intervention. The system can also check the parameter one or more times prior to the event time to confirm that the rate of change of the specific parameter has not changed and the event time has not changed. Where the rate of change of the parameter has changed, the event time can be re-calculated using the new rate of change or as a function of two or more previously determined rates of change. In accordance with some embodiments of the invention, the system can interact with the control system prior to the event time, in order cause a change in the environment or the operation of the machine prior to the specific parameter coming close to the threshold level.

In accordance with some embodiments, the system can determine a measure of degree to which the control system can change the environment or the operation of the machine. For example, the system can determine the number of degrees to increase or decrease the set-point temperature of thermostat, instead of simply increasing the temperature by a predefined value (e.g., raise the temperature 5 degrees). In another example, the system can determine a change in direction and/or velocity of a motor vehicle. In accordance with some embodiments of the invention, the system can take into consideration the operational characteristics of the system or machine being controlled. In accordance with some embodiments of the invention, the system can provide information wirelessly about biometrics, body movement, and physiology to an implanted device (e.g. pacemaker, deep brain stimulator, vagus nerve stimulator, tibial nerve stimulator, spinal cord stimulators, peripheral nerve stimulators, transcranial magnetic stimulators, drug infusion pumps), which in turn, can tune its mode of operation based on this information. For example, for a deep brain stimulation device, the amount of electrical current and stimulus frequency can be configured according to the amount of tremor recorded by the wearable system. The settings that give rise to the lowest prevalence of tremors would be preferentially programmed as part of the feedback loop. In another example, for a thermostat, the amount of time needed to heat up or cool down the room to a specific temperature given the existing environmental conditions can be determined so that the heating or cooling action can be initiated prior to the event deadline, so the desired temperature occurs at or before the event deadline. Similarly, for a motor vehicle, the system can determine the time to initiate the turn (e.g., to avoid hitting an obstacle) or initiate stopping (e.g., to account for stopping distance due to road conditions).

These and other capabilities of the invention, along with the invention itself, will be more fully understood after a review of the following figures, detailed description, and claims.

### BRIEF DESCRIPTION OF THE FIGURES

The accompanying drawings, which are incorporated into this specification, illustrate one or more exemplary embodiments of the inventions and, together with the detailed description, serve to explain the principles and applications of these inventions. The drawings and detailed description are illustrative, and are intended to facilitate an understanding of the inventions and their application without limiting the scope of the invention. The illustrative embodiments can be modified and adapted without departing from the spirit and scope of the inventions.

FIG. 1 is a block diagram of a system according to some embodiments of the invention.

FIG. 2 is a block diagram of a system according to some embodiments of the invention.

FIG. 3 is a block diagram of a system according to some embodiments of the invention.

FIG. 4 is a block diagram of a sensing device according to some embodiments of the invention.

FIG. 5A shows a graph of raw chest accelerometer sensor data where the high frequency component corresponds to heart beats and low frequency component corresponds to the respiration signal. FIGS. 5B & 5C show the accelerometer data after low-pass filtering, which isolates the respiration signal.

FIG. 6A shows an expanded graph of raw accelerometer sensor data of FIG. 5A and FIGS. 6B & 6C show the accelerometer data after low-pass filtering, which isolates the respiration signal.

### DETAILED DESCRIPTION OF PREFERRED EMBODIMENTS

The present invention is direct to systems and methods for modifying the operation or condition of one or more environments or systems as function of one or more sensed conditions of one or more people or subjects. In accordance with the invention, one or more people and/or subjects can be monitored by one or more sensing devices that indicate one or more conditions of some or all of the people and/or subjects. The conditions can include physical conditions, such as location and motion of the person or subject or a part of the person or subject. The conditions can include physiologic or biologic conditions, such as the mechanical, physical, thermal and/or biochemical aspects of the biologic and physiologic functions and/or processes of the person or subject.

In accordance with some embodiments of the invention, the sensed condition information can be used to modify the operation of the system (e.g., a device or set of devices), for example, to cause a computer program, function or process to be executed or to change the flow of an executing program, function or process. In one example, a motion sensor (e.g., an accelerometer) could detect motion characteristic of running or walking and as a result, the system could cause a step tracking computer program, function or process to be executed to count the number of steps taken and the distance traveled.

In accordance with some embodiments of the invention, the sensed condition information can be used to modify the operation of the system (e.g., a device or set of devices), for example, to cause other sensors to be activated so that their data can be used as part of newly started or an ongoing computer program, function or process for monitoring the person or subject. In one example, a temperature sensor could detect a rise in the person or subject' temperature (e.g., above threshold or steady state body temperature) and as a result, the system could activate sensors to detect heart rate (e.g., EKG) and/or respiration and as a result, begin to monitor heart rate and/or respiration as part of a newly started or ongoing health monitoring program, function or process. Similarly, the rise in temperature above the designated threshold could trigger an implantable device to reconfigure from idle mode to stimulatory mode based on wireless signal transmission from wearable system to implantable device.

In accordance with some embodiments of the invention, the sensed condition information can be used to cause the system (e.g., a device or set of devices) to communicate with one or more other systems, resulting in a change in operation of these other systems. For example, the system can send a signal (e.g., wired or wireless) to a remote system and the signal can cause the remote system to change its operation or the operation of a system under its control (e.g. an implantable device such as a pacemaker or a deep brain or spinal cord stimulator). In another example, the system can detect a temperature drop or an increase in of the person or subject and the system can send a signal to a thermostat and the thermostat can cause a heating system to turn on and/or raise the temperature in the environment.

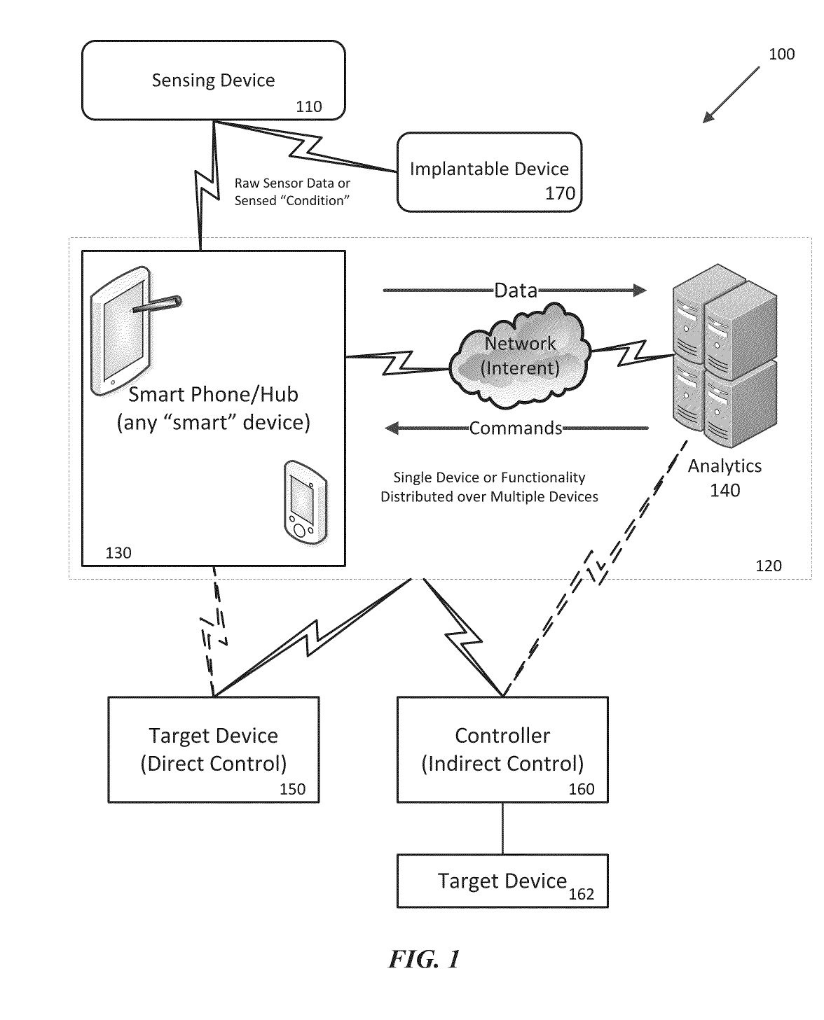

FIG. 1 shows an example of a system 100 according to some embodiments of the invention. In this embodiment, the system 100 can include one or more sensing devices 110, a smart phone or hub 130, and target device 150 and/or controller 160. An optional analytics system 140 can also be connected to the system 100. The controller 160 can be connected to and used to control a target device 162, indirectly. In accordance with some embodiments, one or more sensing devices 110 can communicate with an implantable device 170 implanted in the body of person or subject.

The sensing device 110 can be any device capable of detecting or measuring physical, physiologic or biologic functions and more than one sensing device can included in the system 100. Each sensing device 110 can be configured with one or more controllers or microcontrollers, such as a low power system on a chip microcontroller, associated memory and a power source, such as a battery. The controller can be configured to run one or more digital signal processing algorithms and/or raw data signal processing algorithms. Each sensing device 110 can include one or more sensors such as accelerometers, gyroscopes, temperature sensors, light sensors (e.g., visible and invisible light), sound sensors, bio-potential electrodes (e.g., ECG, EMG, EEG), and other sensors. Each sensing device 110 can be configured to send sensor data to the smart phone or hub 130 or the implantable device 170. The sensor data can include raw sensor signal data, processed sensor signal data (e.g. filtered, scaled, segmented), signal features (e.g. dominant frequency, range, root mean square value) and algorithm output (e.g. fall detection alarm, tremor score, sleep quality, posture quality). The sensor data can include other information, such as metadata (e.g., information about the sensor device, the date, the time, the type and the scale or units of the sensor data).

Some examples of sensors and types of sensor data include, but are not limited to, dry and gel-enhanced electrodes and electrode arrays for measuring electrocardiogram waveforms, heart rate, heart rate variability, electromyography from distinct muscle groups (e.g. tibialis anterior muscle), electroencephalograms, electro-oculagrams. Strain gauges for measuring pulse waveforms from superficial arteries and respiration patterns. Piezoelectric sensors and actuators for mechanical energy harvesting and pulse waveform measurements. Temperature sensors, such as thermal couples and thermistors (for measuring core and skin surface temperature), optical sensors and/or photodetectors (for ultraviolet, visible light analysis, and/or colorimetry analysis), pH sensor, bioanalyte sensor (e.g. potassium, sodium, calcium, glucose, hormones, proteins), chemical/gas sensor (pollutants, deadly gases, mercury), microfluidic sensors for capturing and analyzing (e.g., composition and volume) skin secretions including perspiration and oils. Other sensor data can include derivative sensor data derived (e.g., derivative data) from the raw sensor data over time or frequency.

The processed sensor data can be derived from the raw sensor data by various well known processes to remove noise or to characterize sets or units of raw sensor data (e.g., into features, tokens and/or messages). The sensing device 110 can include a processor and associated memory and execute one or more computer programs that collect sensor data on a periodic basis. The sensing device 110 can include a communication system that enables the raw sensor data or the processed sensor data to be transmitted to a remote device or system, such as the smart phone or hub 130. The communication system can be adapted to provide wired or wireless communication with a remote device, such as the smart phone or hub 130.

Each sensing device 110 can take many forms, including, for example, a flexible or stretchable conformable sensing device that can be adhered to the skin, a bracelet or strap that can be worn on the body, an article of clothing or a pad or plate that can be positioned against or adjacent the body. The sensing device 110 can detect and measure (either directly or indirectly) the physical motion of the person or subject. The sensing device 110 can include sensors that detect and measure (either directly or indirectly) temperature, ambient environmental temperature as well as the temperature of the person or subject (e.g., core body and/or skin surface). The sensing device 110 can include sensors that detect and measure (either directly or indirectly) the pulse, blood pressure, galvanic skin response, and/or blood oxygen of the person or subject. The sensing device can include sensors that detect and measure bio-potentials (e.g., EKG, EMG and EEG signals), strain, surface body temperature, core body temperature, salt concentrations in sweat, sweat loss rate, blood micronutrient levels, glucose concentration in sweat, visible/infrared/ultraviolet radiation, contact pressure, barometric pressure, skin strain, skin modulus, images of sub-dermal structures using ultrasound transducers from the person or subject. The sensors can include optical sensors that detect changes is light produced, light reflect and/or light transmitted through a sample being sensed, for example, as result of a chemical reaction. The sensors can include electrochemical sensors that detect changes in electrical potential, resistance and/or impedance as result of a chemical reaction, for example, the change in electrical signal resulting from an analyte (e.g., blood, sweat, or other bodily fluids) interacting with a sensor substrate. The sensing device can contain actuators to deliver electric current (electric fields) to transdermally direct nanoparticles through skin (e.g. pharmacological agents), LED arrays (blue and near infrared light) to deliver photo-activation therapy to skin.

The sensing device 110 can sample the output of one or more sensors on a periodic basis (e.g., at 1 Hz, 5 Hz, 10 Hz, 60 Hz, or more) and, if necessary, convert the signals into digital data. The digital data can be buffered, stored, transmitted and/or streamed to one or more remote devices.

The implantable device 170 can be any known implantable device that can communicate with the sensing device 110 (and/or optionally, the smart phone or hub 130) and sense a condition of the person or subject from inside the body and/or apply a treatment or therapy to the body. Implantable devices 170 can include pacemakers, deep brain stimulation devices, vagus nerve stimulators, tibial nerve stimulators, spinal cord stimulators, peripheral nerve stimulators, transcranial magnetic stimulators, and/or drug infusion pumps (e.g., insulin pumps for delivering insulin to blood vessels or other organs). The implantable device 170 can receive information from the sensing device 110 and change its operation (e.g., adjust the pacemaker heartbeat, adjust the level of brain or nerve stimulation, and/or adjust the drug infusion levels). In accordance with some embodiments, the sensing device 110 can communicate with one or more implantable devices 170 through an intermediate device such as smart hub 130 or an external controller.

The smart phone or hub 130 can be a smart phone or other computerized device that can communicate with the sensing device 110 using any wired or wireless communication band (e.g., Bluetooth, WiFi, ZigBee, WMTS, cellular data, and industrial, scientific, and medical (ISM) band communications). The sensor device 110 and the smart phone or hub 130 can use an industry standard communication protocol or a proprietary communication protocol. The smart phone or hub 130 can include a processor and associated memory that can receive the raw sensor data or the processed sensor data from the sensing device 110 and store it in memory for further processing or for communication to a remote system for further processing, such as analytics system 140. The smart phone or hub 130 can include one or more sensors (e.g., accelerometer, GPS, temperature, light). The smart phone or hub 130 can include a network interface (e.g., wired such as Ethernet or wireless such as WiFi or 3G, 4G, 4G LTE mobile data) that enables the smart phone or hub 130 to communicate other smart phones, computers, and systems, such as analytics system 140 and other sources of data and information. In accordance with the invention, the smart phone or hub 130 and/or the analytics system 140 can further analyze the sensor data using analytics algorithms that either process the sensor data by itself or in combination with other available data. In accordance with some embodiments of the invention, the smart phone or hub 130 can analyze the sensor data and as a function of at least the sensor data, directly communicate with another device to control that device. For example, the smart phone or hub 130 can receive sensor data (either from the sensing device 110, its own internal sensor, or both) indicating the level of illumination in an environment, such as a room, and as a function of the sensed illumination data, directly turn on or off or dim one or more lights in the room.

In accordance with some embodiments of the invention, the smart phone or hub 130 can analyze the sensor data and as a function of at least the sensor data, indirectly communicate with another device through an interface, such as separate control system in order to control that device. For example, the smart phone or hub 130 can receive sensor data indicating the ambient temperature level in an environment, such as a room or the person or subject, and as a function of the sensed temperature data, directly control the heating and/or cooling (e.g., turn the HVAC system on or off, or adjust the thermostat set-point temperature up or down) for the room. In another example, the smart phone or hub 130 can receive sensor data indicating a physical or biological condition (e.g., motion, heart rate, respiration rate, temperature) of a person and either send some or all of this data to a gaming console, wherein the program running on the console changes at least a portion of the virtual world presented by the gaming console as a function of the physical and/or biological data received or the smart phone or hub 130 can process the physical and/or biological data and produce commands or instructions that are sent to control a gaming console and optionally, act as inputs to the program running on the game console to change at least a portion of the virtual world presented by the gaming console as a function of the commands or instructions received.

In accordance with some embodiments of the invention, the smart phone or hub 130 can send the raw sensor data or the processed sensor data (or both) to a remote analytics system 140 that can process and analyze the sensor data and the analytics system 140 can communicate directly or indirectly with other devices to control them and the environment.

In accordance with some embodiments of the invention, the smart phone or hub 130 together with remote analytics system 140 can process and/or analyze the raw or processed sensor data, optionally in combination with other data from other sensors or stored data, weather data, or date and time information, to determine one or more actions. The actions can include communicating with a target device 150 to control it directly or communicating with a remote controller 160 that controls the target device 162.

In accordance with some embodiments of the invention, the sensing device 110 can send the sensing data (e.g., raw sensor data, processed sensor data) to the implantable device 170, the target device 150 or the controller 160 and the implantable device 170, the target device 150 or the controller 160 can analyze the sensing data and determine a change in the operation of the implantable device 170, the target device or the controller 160 as a function of the sensing data. For example, the implantable device can raise or lower a stimulation level or drug therapy dosage level of the implantable device 170 or the target device 150 as a function of sensed condition such as heart rate, respiration rate, or glucose levels. A change in operation can include turning the device on or off, increasing or decreasing a level of operation, or changing profile of operation (e.g., a defined pattern of operation over time, such as stimulate at a first level for predefined period and then turn off or change the stimulation level to second level for a period of time).

In accordance with some embodiments of the invention, the sensing device 110 can analyze the sensor data and determine a change is need in the operation or configuration of the implantable device 170, the target device 150 or the controller 160. The sensing device 110 can also send a query instruction to the implantable device 170, the target device 150 or the controller 160 to obtain status information about its operating status and configuration and then determine based upon the status information and sensing data, determine a change in the operation or configuration of the implantable device 170, the target device 150 or the controller 160. After the change is determined, the sensing device 110 can send a command (or set of command) or an instruction (or set of instruction) to the implantable device 170, the target device 150 or the controller 160 to cause the change in operation to occur. In accordance with some embodiments, in addition to the commands or instructions, the sensing device 110 can also send sensor data to the implantable device 170, the target device 150 or the controller 160.

In accordance with some embodiments of the invention, the sensing device 110 can send the sensing data (e.g., raw sensor data, processed sensor data) and/or the queries, commands and/or instructions to the smart phone or hub 130 and the smart phone or hub 130 can send the sensing data and/or the queries, commands and/or instructions to the implantable device 170, the target device 150 or the controller 160 to change the configuration or operation of the implantable device 170, the target device 150 or the controller 160. In accordance with some embodiments, the smart phone or hub 130 can send the sensing data and/or the queries, commands and/or instructions along with hub data (e.g. date, time, environmental temperature, weather conditions, geolocation, accelerometer/gyroscope data, sound, images, and trends of any of the foregoing) to an analytics system 140 which can analyze the sensing data and/or the queries, commands and/or instructions and the hub data and determine operational changes for the implantable device 170, the target device 150 or the controller 160. The analytics system 140 can instruct the smart phone or hub 130 to query the implantable device 170, the target device 150 or the controller 160 for status or configuration information and use the configuration information along in combination with other data to determine operational changes in the implantable device 170, the target device 150 or the controller 160. The analytics system 140 can instruct the smart phone or hub 130 to send sensing data and/or commands and/or instructions to the implantable device 170, the target device 150 or the controller 160 to change its operation. In some embodiments, the sensing data and/or commands and instructions can be modified versions or completely different from the sensing data and/or commands or instructions that were received from the sensing device 110.

As shown in FIG. 1, in accordance with some embodiments, the analytics functionality can be distributed over a one or more smart phones or hubs 130 in a network or cluster configuration to form a distributed processing system 120 to provide for distributed processing of the sensor and, optionally, other data. In accordance with some embodiments, the analytics functionality can be distributed over the smart phone or hub 130 and one or more computer systems or clusters (e.g., other smart phones or hubs 130, and/or analytics computer systems 140), in a distributed network or cluster system configuration 120 to provide for distributed processing of the sensor and, optionally, other data. Each of the computer systems that make up the cluster can communicate using wired cluster interconnect technologies and/or wireless communication technologies (e.g., Ethernet, WiFi, mobile data, such as, GSM, 3G, 4G, and 4G LTE) or other network communication technologies. The network can include networking equipments, such as, one or more wires, switches, hubs, wireless access points, and routers to enable communication between the devices and systems.

In accordance with some embodiments of the invention, the smart phone or hub 130 can be configured to communicate directly with one or more target devices 150 using wired or wireless communication (e.g., infrared, Ethernet, Bluetooth, WiFi, ZigBee, WMTS, cellular data, and industrial, scientific, and medical (ISM) band communications). In accordance with some embodiments of the invention, the smart phone or hub 130 can be configured to communicate directly with one or more controllers 160, using wired or wireless communication (e.g., infrared, Ethernet, Bluetooth, WiFi, ZigBee, WMTS, cellular data, and industrial, scientific, and medical (ISM) band communications). The controllers 160 can be controlled using an open or proprietary interface or application programming interface (API) to control the target device 162.

The analytics system 140 can include one or more computers that are configured to receive the sensing data. The sensing data can be transmitted by the smart phone 130 to the analytics system 140 over a public or private network. In accordance with some embodiments, the hub 130 acts a gateway that forwards the sensor data to the analytics system 140 according to predefined instructions or configuration. The analytics system 140 can be, for example, a big data server (e.g., based on Hadoop, or another analytics engine) that can receive, store and analyze the sensor data according to a predefined analytical method or process. In accordance with some embodiments, as a result of the predefined analytical method or process, the analytics system 140 can generate one or more commands and/or data and send one or more of those commands and/or data to the smart phone or hub 130, a target device 150 or a controller 160. The commands can be used to control or change the operation of the smart phone or hub 130, a target device 150 or a controller 160.

In accordance with some embodiments, the smart phone 130 can send one or more commands (e.g., an instruction to do something or perform some function or operation, or an acknowledgement that a function or operation has started or completed) and/or data (e.g., sensor data, user data, and environmental data) to the analytics system 140. The analytics system 140 can interpret and respond to the commands, for example, to retrieve data or process data or change the way the analytics system 140 processes the data. The response can include a command (e.g., an acknowledgement or instruction) and/or data (e.g., data or information requested, results of an analysis or other sensor data). The smart phone or hub 130 can use the data for further analysis by algorithms on the smart phone or hub 130 or to determine whether one or more commands and/or data should be sent to a target device 150 or a controller 160.

In accordance with some embodiments of the invention, the target device 150 can include a device that can communicate directly with the smart phone or hub 130. Thus, the target device 150 could include, for example, a light switch, an appliance (e.g., TV, refrigerator, dishwasher, or washing machine), a garage door opener, a door lock, a manned or unmanned motorized vehicle (e.g., car, truck, train, boat or airplane), a computer, a programmable controller, a sound system, an environmental control system (e.g., HVAC system, heating system, cooling system, humidifying system, dehumidifying system), a home automation system, a virtual reality console, a gaming console, and a communication system (e.g., voice/telephone, text messaging, email, facsimile, and chat). In accordance with some embodiments of the invention, the target controller 160 can include, for example, a home automation controller (e.g., to control target devices 162, such as lights, HVAC, garage doors, door locks, appliances, and sound systems), an HVAC controller (e.g., thermostat), home entertainment system, a dispatch system (e.g., dispatching motorized vehicles, people and/or services), and a motor vehicle control system (e.g., controlling vehicle operation, including direction and navigation, safety, and vehicle environmental control). In accordance with some embodiments of the invention, the target controller 160 can hospital room or surgical suit control system that can control the environment of the hospital room or surgical suite and/or control the operation of equipment in the hospital room or surgical suite (e.g., respiration equipment, dialysis equipment, anesthesia equipment, infusion pumps).

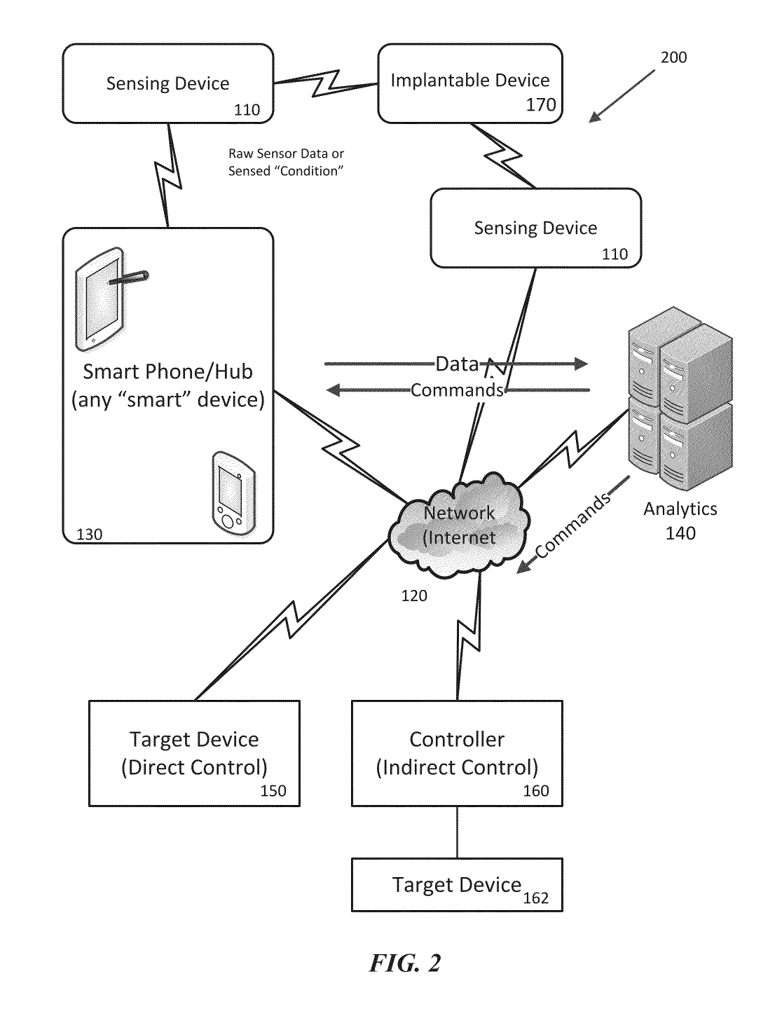

FIG. 2 shows a system 200 similar to system 100 according to some embodiments of the invention. In this embodiment, the system 200 can include one or more sensing devices 110, a smart phone or hub 130, and target device 150 and/or controller 160. An optional analytics system 140 can also be connected to the system 100. The smart phone or hub 130 can be configured to communicate with the target device 150, the controller 160 and optional analytics system 140 over a common network 120, such as a LAN, or a WAN, such as the interne. The controller 160 can be connected to and used to control a target device 162, indirectly. System 200 can include many of the same components as system 100 and operate similar to system 100.

The primary difference between system 200 and system 100 shown in FIG. 1 is that a common network 120 is used to communicate between each of the components of the system 200. While one sensing device 110 is shown as being connected directly to the smart phone or hub 130, in an alternative embodiment of the invention, one or more the sensing devices 110 (e.g., one or more worn by the person or subject and optionally, one or more not worn by the person or subject) could be connected to the smart phone or hub 130 through the common network 120. The smart phone or hub 130 can communicate with one or more sensing devices 110, one or more analytics systems 140, a target device 150 and a controller 160 over the common network 120.

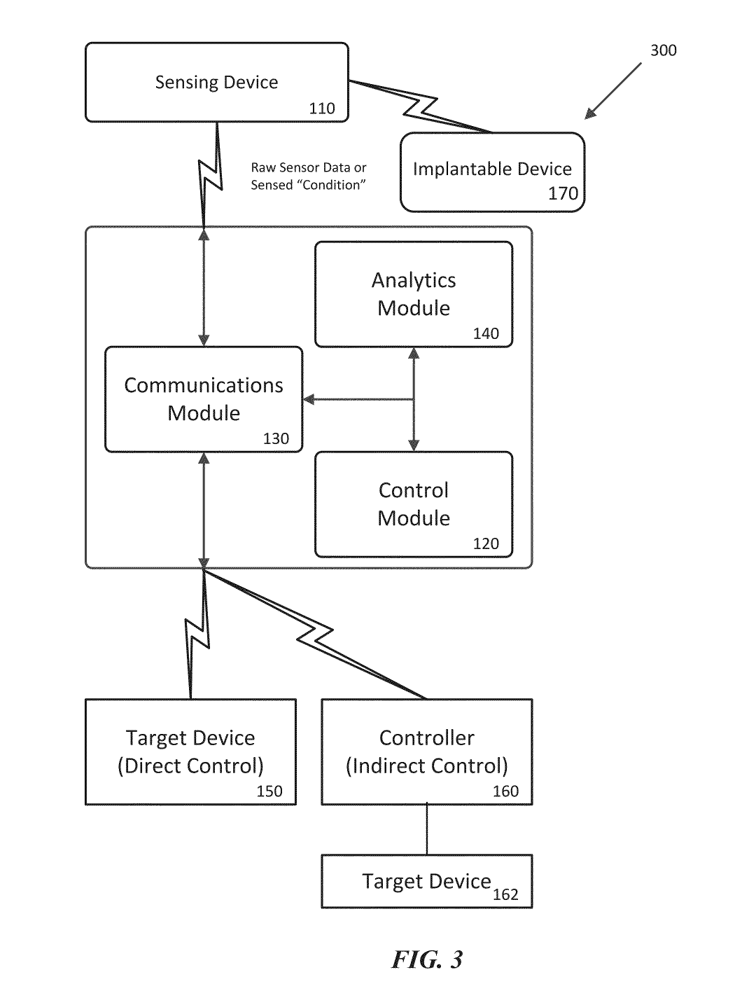

FIG. 3 shows a system 300 similar to systems 100 and 200 according to some embodiments of the invention. In this embodiment, the system 300 can include one or more sensing devices 110, a control module 120, a communications module 130, an optional analytics module 140, and a target device 150 and/or a controller 160. The control module 120, a communications module 130, and analytics module 140 can be incorporated into a common device (e.g., one device that performs all the functionality, such as a computer or smart phone) or implemented as standalone components connected by a common communication channel, such as shared bus or network. The controller 160 can be connected to and used to control a target device 162, indirectly. System 300 can include many of the same components as systems 100 and 200, and operate similar to systems 100 and 200.

As shown in FIG. 3, the control module 120 can be connected to and communicate with the communications module 130 and the analytics module 140. The communications module 130 facilitates the communication of sensor data and commands between the sensing device 110 and control module 120 and/or the analytics module 140. The communications module 130 also facilitates the communication of messages and/or commands between the control module and/or the analytics module 140 and one or more target devices 150 and/or one or more controllers 160.

In operation according to some embodiments of the invention, sensor data from the sensing device 110 is received by the communications module 130. The sensor data can optionally be stored in memory. The sensor data is sent to the control module 120 for subsequent processing. In accordance with some embodiments, the sensor data is processed according to one or more algorithms or processes which determine whether one or more conditions relating to the sensor data meet one or more specified criteria. This can be accomplished by comparing one or more of the conditions to a predefined threshold and taking an action (or not taking an action) as a function of the comparison. For example, if a subject's body temperature is below a first predefined threshold and the environmental temperature is below a second predefined threshold, then the system can increase the environmental temperature by a first incremental amount by sending a command to the thermostat or the home automation controller to cause the heating system to turn on and raise the environmental temperature by a predefined amount or cause the air conditioning system to turn off, to allow the environment to warm up. If the subject's body temperature does not increase after the environmental temperature is increased by the first predefined amount, the system could send a second command to the thermostat or the home automation controller to cause the heating system to turn on (if necessary) and raise the environmental temperature by a second predefined amount (which could the same as the first) or cause the air conditioning system to turn off or stay off, to allow the environment to warm up. After the passage of a set period of time or number of heating cycles, if the subject's body temperature does not rise, the system could send an alert message to a family message or care provider.

In accordance with some embodiments, the sensor data can be processed according to one or more algorithms or processes which determine whether one or more conditions relating to the sensor data meet one or more specified criteria (e.g., threshold values) or are likely to meet one or more specified criteria in the future. This can be accomplished by extrapolating existing data or analyzing trends in the data, to determine a predicted value for one or more conditions in the future and comparing the predicted value to a predefined threshold. For example, if a subject's body temperature is dropping as demonstrated by a recorded drop in temperature over time, based on the determined rate of change, the algorithm can determine the warning time, e.g., the amount of time it will take for the subject's body temperature to drop below a predefined threshold under the current environmental conditions (e.g., the current ambient temperature). Using other information, such as the heating efficiency of the heating system or warming rate (e.g., when the air conditioning is turned off), the algorithm can determine a warm-up time, e.g., how long it will take the heating system to warm up (e.g., raise the temperature of) the environment by a first amount. If the warning time is greater than the warm-up time, the system 100, 200, 300, can wait some time less than the difference between the warning time and warm-up time and check the subject's body temperature. Alternatively, the system 100, 200, 300, can begin to raise the heat (or keep the air conditioning off), and monitor for any changes in the subject's body temperature and increase or decrease the environmental temperature depending upon whether the subject's body temperature continues to drop or whether it begins rise.

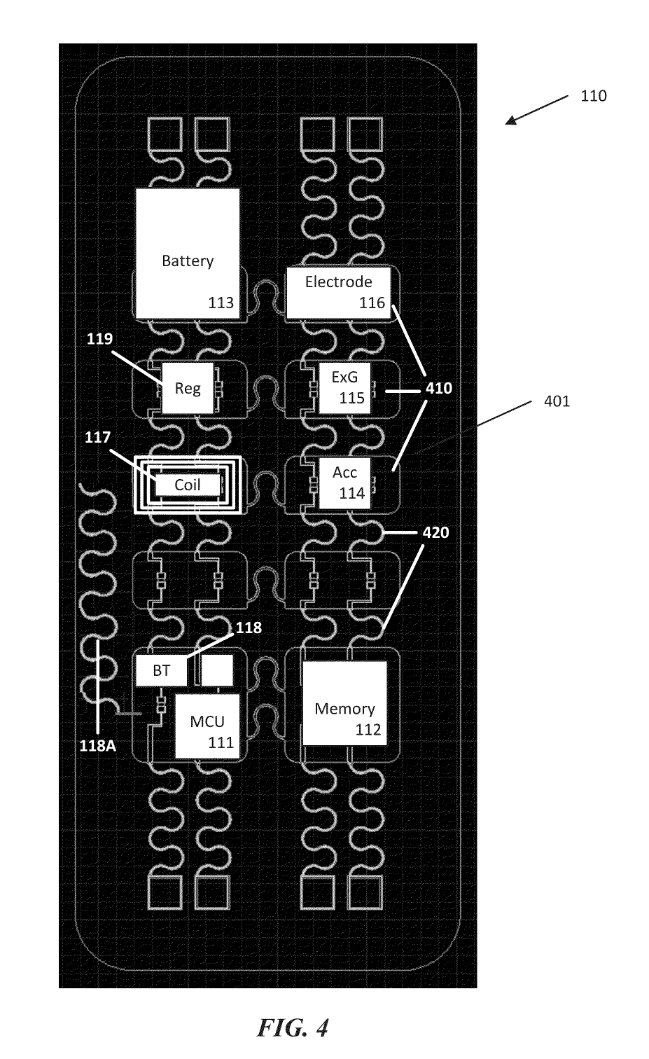

FIG. 4 shows one embodiment of a sensing device 110 according to the invention. In accordance with some embodiments of the invention, the sensing device 110 can include a plurality of components mounted on device islands, 410 wherein each device island 410 can be electrically connected to an adjacent device island 410 by a flexible and/or stretchable interconnect 420, enabling the sensing device to flex and stretch and conform to irregular surfaces, such as those of the body of a person or subject. The sensing device 110 can be encapsulated in a flexible or stretchable material, such as, silicone or PDMS. The sensing device 110 can include an adhesive material that enables the sensing device to adhere to the skin of a person or subject or the surface of an object. The sensing device 110 can optionally include one or more user interface components, such as buttons, lights (e.g., LEDs), displays, speakers or vibrators that enable a user or person to interact with the device using visual, audible and sensory cues. These user interface components can be used to provide operational, configuration, and biometric performance feedback to a user directly, such as, through visual and tactile output capabilities via LEDs and vibration motors.

The sensing device 110 can include a processor 111 and associated memory 112 and a battery 113 which serves as a power source. An induction coil 117 can be used to charge the battery 113. The sensing device 110 can include one or more sensors, including an accelerometer 114, an ExG sensor 115 and one or more electrodes 116. The sensing device 110 can also include wireless transceiver 118 (e.g., such as Bluetooth™, WiFi, mobile data) and an antenna 118A to enable the sensing device 110 to communicate with smart phone or hub 130.

In accordance with some embodiments, the memory 112 can store one or more computer programs, including an operating system (e.g., Linux) as well as one or more application programs, functions and processes that can be used to control the operation of the sensing device 110. One or more programs, functions or processes can be used to collect accelerometer data, which includes motion and acceleration information in 1, 2 or 3 dimensions as well as temperature data. One or more programs, functions or processes can be used to collect bio-potentials in the form of ExG data from the ExG sensor. The ExG data can include data representative of at least one of the following bio-potential signals: electrocardiogram (e.g., EKG or ECG) signals, electromyogram (e.g., EMG) signals or Electroencephalogram signals (e.g., EEG), depending on how the one or more programs, functions or processes configures the ExG sensor 115. The sensing device 110 can include one or more electrodes 116 that can be placed in contact with the skin to receive these signals. In accordance with some embodiments of the invention, the EKG data can be used to determine heart rate and heart rate variability as well as recovery rate and the EMG data can be used to determine muscle activation.

In operation, the sensing device 110 can be configured using one or more programs, functions or processes to collect raw sensor data and store the data in memory 112. In accordance with some embodiments, one or more programs, functions or processes running on the processor 111 can process and/or analyze the raw sensor data and generate processed sensor data, for example, by filtering the raw data to remove noise and/or artifacts and/or to normalize the raw sensor data. In accordance with some embodiments, the raw sensor data and/or the processed sensor date can be further processed by computing descriptive analytics (e.g., minimum values, maximum values, mean values, median values, mode values, standard deviation and variance values, and higher moments such as kurtosis) on one or more sets of samples of the data, and comparing such values against the comparable values of a larger cohort of relevant individuals, or against prior measurements collected on the same individual. In accordance with some embodiments, the raw sensor data or the processed sensor data can be further processed to extract specific features or characteristics of the signal like the dominant frequency, range, root mean square value, correlation coefficient, heart rate, respiration rate, cadence etc. The features can be further processed using one or more algorithms (e.g. decision tree, state machine, and/or linear/logistic regression) to detect or predict events (e.g. falls, activity type, seizure, tremor) or to detect or predict status (e.g., state of mind, mental condition and/or attitude). In accordance with some embodiments, the raw sensor data can be converted to tokens or symbols representative of two or more raw sensor data values. The raw sensor data can be processed in real time as it is received from the sensor element or it can be processed in blocks after a predefined number of raw sensor data values are received. The raw data and the processed data can be stored in memory 112, until it is transmitted to a remote device.

The sensing device 110 can process the data to generate one or more higher order biometrics, by processing the raw data to determine, for example, activity type detection, activity-specific or body location-specific performance indicators, gesture recognition, posture quality, and sleep quality. The sensing device 110 can receive and process external commands which cause the device to modify its configuration and/or operation for collection, processing, and reporting of sensor data, including turning on or off various sensor combinations, changing sampling rates and measurement ranges, modifying buffering and filtering schemes, and applying different digital signal processing and algorithms to raw sensor output to produce different streams of data and/or different sets of higher order biometrics around activity tracking, activity performance, and activity quality data. Based on the biometrics determined and/or other data, the sensing device 110 can, based on an algorithm or set of rules, select a sensing modality which is optimal for a particular activity or on-body location that has been detected, and automatically modify its configuration and/or operation for collection, processing, and reporting of sensor data, including turning on or off various sensor combinations, changing sampling rates and measurement ranges, modifying buffering and filtering schemes, and applying different digital signal processing and algorithms to raw sensor output to produce different streams of data and/or different sets of higher order biometrics around activity tracking, activity performance, and activity quality data.

In accordance to some embodiments of the invention, when the sensing device 110 is connected using, for example, the wireless transceiver 118 (e.g., Bluetooth™, WiFi or Zigbee) to the smart phone or hub 130 or implantable device 170, the raw sensor data and/or the processed sensor data can be transmitted using the wireless transceiver 118 to the smart phone or hub 130 or implantable device 170 and stored in the memory of the smart phone or hub 130 or an implantable device 170. In accordance with some embodiments of the invention, the sensor data can be transmitted by the smart phone or hub 130 or implantable device 170 to the analytics system 140 for long-term storage and further analysis.

The systems 100, 200, 300 can be configured to enable many different data flows. In accordance with some embodiments of the invention, the raw data or processed sensor data (metrics) can flow from the sensing device 110, through the smart phone or hub 130 or implantable device 170, to the analytics system 130 or a the data storage system associated with the analytics system 130. The sensor data (e.g., raw or processed) can be pre-filtered, conditioned, manipulated, or combined with other data within the smart phone or hub 130. The sensor data (e.g., raw or processed) can also be filtered, conditioned, manipulated, or combined with other data within the data storage and analytics system 140, and can be used to tune the electrical/neuromodulation and/or drug therapy delivered by an implantable device 170 (e.g., a nerve stimulation device or drug-delivery system).

In accordance with some embodiments of the invention, processed sensor data or other data can flow from the data storage and analytics system 140 through the smart phone or hub 130 or implantable device 170 and back to the sensing device 110. Processed data (e.g., commands, control instructions, or higher order information, such as, software and algorithms for system upgrades and updates) can flow from the data storage and analytics system 140 to the smart phone or hub 130 or implantable device 170, and through the smart phone or hub 130 or implantable device 170 to the sensing device 110. The data can be filtered, interpreted, validated, and/or combined with other data within the smart device. The data can also be filtered, interpreted, validated, and/or combined with other data within the sensing device 110.

In accordance with some embodiments of the invention, the raw data or processed sensor data (metrics) can flow from the sensing device 110 (optionally through the smart phone or hub 130), through the data storage and analytics system 140 to one or more external systems, such as, machines, equipment, implantable device and environmental control systems. Processed data (commands, control instructions, or higher order information, such as, software and algorithms for system upgrades and updates) can flow from the data storage and analytics system 140 to external machines or equipment (e.g., exercise equipment, power tools, motorized vehicles) and/or environmental control systems (such as ambient temperature control system, lighting, or alerting and alarm systems). The data can be filtered, interpreted, validated, and/or combined with other data within the external machine, equipment or environmental control system. The data can also be filtered, interpreted, validated, and/or combined with other data within the sensing device 110.

### Applications

In accordance with some embodiments of the invention, body worn conformable sensors, such as sensing device 110 can be used to quantitatively measure a variety of different health parameters from the human body. These physiologic and biologic parameters include bio-potentials, such as, ECG, EMG and respiration as well as acceleration and angular velocities related to motion. The ECG signals can be used to determine heart rate, heart rate variability and recovery rate. In addition ECG signals can be used to determine abnormalities associated with heart function (e.g. seizures and/or arrhythmias including tachycardias, bradycardias, atrial fibrillation, atrial flutter, and/or supraventricular tachycardias. The EMG signals can be used to determine muscle activation (e.g., contractions, spasms, tremor, rigidity, myopathy, and/or muscular dystrophy). The raw accelerometer signals can be transformed into signal parameters or features such as frequency content in specific frequency bands, acceleration vector amplitudes, acceleration vector direction changes as a function of time, etc. and these features can be correlated with relevant metrics such as heart rate, respiration rate, as well as motion related to walking, running, physical activity, posture, tremors, coughs, snoring, frailty, and falls. The accelerometer signals can also be used to detect and/or measure, for example, seizures, gait and balance problems, steps and/or cadence, energy expenditure (together with heart rate and/or respiration rate), range of motion, and other activity types (e.g., swimming, cycling, rowing, throwing, swinging, kicking, punching, etc.)

These parameters can be used for detection or prediction of medical conditions and/or as an indicator of general health and well-being of an individual. In accordance with some embodiments of the invention, a system that includes a wearable body sensor can be connected to an information gateway (e.g., hub 130) to control a set of external devices nearby the user. The system can act in a coordinated manner to provide responsive action for influencing the wellbeing of an individual. For example, implantable neuromodulation devices 170 can be configured and controlled for delivering therapy according to the signals captured by the wearable body sensor 110.

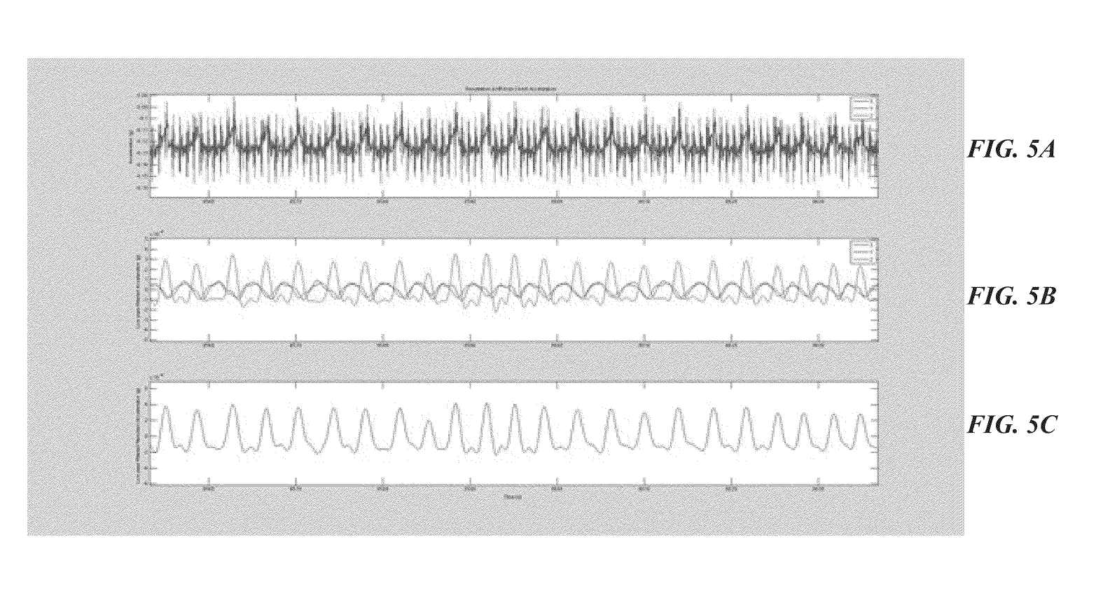

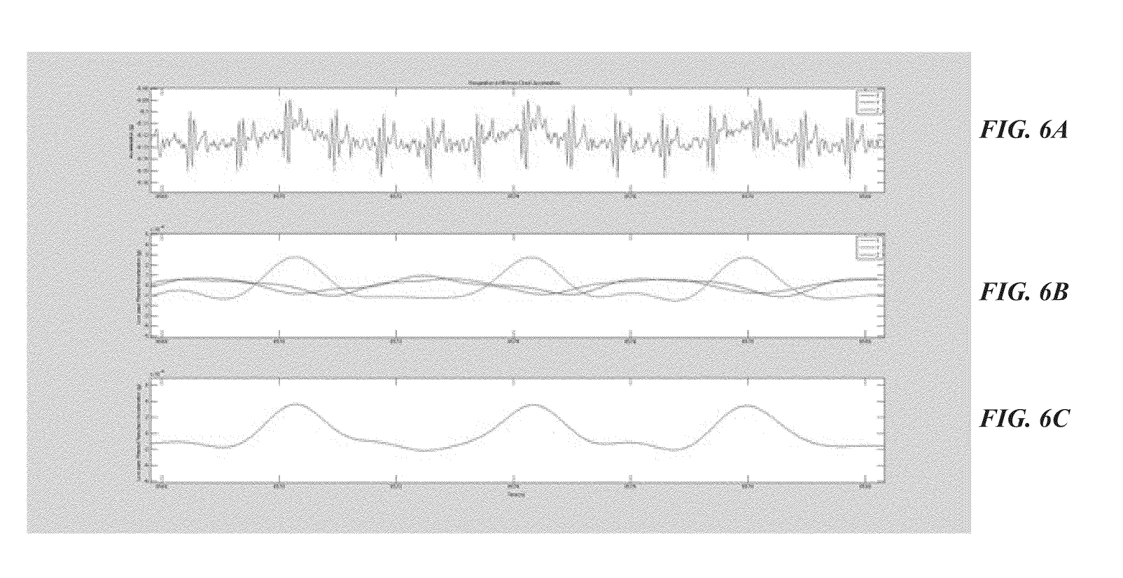

In accordance with some embodiments of the invention, accelerometer data captured by a conformable sensing device 110 affixed to the chest can be used detect and record multiple physiologic signals including heart rate, respiration, coughing at rest, by detecting the mechanical vibrations resulting from the motions of the chest cavity and rhythmic movements of the heart (heart rate) sensed on the chest wall. FIG. 5 shows a comparison of raw accelerometer data (top), and filtered accelerometer data (middle and bottom) that maps directly to breathing frequency and amplitude. FIG. 6 shows an exploded view of raw accelerometer data (top) showing the high frequency heart beat bursts riding on the low frequency breathing pattern envelope and filtered accelerometer data (middle and bottom) showing low-frequency patterns of oscillation, which map to breathing patterns.

In accordance with some embodiments of the invention, heart beat information can be derived from the high frequency portion of the accelerometer signal, and respiration information can be derived from the low frequency portion of the accelerometer signal. For example, to derive heart rate information, the raw accelerometer data can be filtered through a band pass filter with a high pass cutoff frequency of 2 Hz and low pass cutoff frequency of 45 Hz. Next, the resultant of the X, Y and Z axes is determined by taking the square root of the sum of square of each axis. To amplify the high frequency components, the signal is then differentiated. The differentiated signal is then processed by a state machine with an adaptive threshold to detect the heart beats and calculate the heart rate using a Pan-Tompkins or similar algorithm. See, Pan, Jiapu; Tompkins, Willis J., “A Real-Time QRS Detection Algorithm,” Biomedical Engineering, IEEE Transactions on, vol.BME-32, no.3, pp.230,236, Mar. 1985, which is hereby incorporated by reference.

For example, to derive the respiration rate information, the raw accelerometer signal can be filtered using a low pass filter having a cutoff frequency of 2 Hz. The respiration rate can be estimated based on the method described in the following publication: A. Bates, M. J. Ling, J. Mann and D. K. Arvind “Respiratory Rate and Flow Waveform Estimation from Tri-axial Accelerometer Data”, Proc. Int. Conf. on Wearable and Implantable Body Sensor Networks, pages 144-150, Singapore, June 2010

The flexible or stretchable sensing device 110 according to the invention can be used for sleep quality analysis and treatment. Sleep quality analysis (e.g., sleep studies) typically require multiple biometric data streams to determine sleep states including awake state, light sleep, deep sleep and rem sleep. In the prior art, these sleep studies tend to collect multiple streams of data from patients, including respiration rate/patterns, heart rate/heart variability, roll over frequency, motion, EEG, and other parameters using wired sensors to evaluate sleep quality. In accordance with some embodiments of the invention, one or more flexible or stretchable conformable sensing device 110 according to the invention can be used to monitor multiple sleep parameters—respiration, heart rate, roll-over frequency and temperature—with an accelerometer sensor housed in an extremely soft and stretchable package that is sufficiently sensitive to sense very small mechanical perturbations caused by breathing and heart beats. In accordance with some embodiments of the invention, a single conformable sensing device 110 can be adhered to the chest to detect respiration, heart rate, roll-over frequency and temperature using just an accelerometer sensor. The sensor data can be transmitted wirelessly to the smart phone or communications hub 103 that stores the accelerometer sensor data and can forward the sensor data to a remote computer system for subsequent analysis. In accordance with some embodiments of the invention, two or more conformable sensing devices 110 can be used. A first conformable sensing device 110 can be adhered to the chest to detect respiration, heart rate, roll-over frequency and temperature using just an accelerometer sensor and a second conformable sensing device 110 can be adhered to the head and using the ExG sensor detect EEG data in addition to the accelerometer data collected by the first conformable sensing device 110.

The accelerometer data and optionally, the EEG data can be processed and analyzed by the smart phone or hub 130, analytics module 140 and/or implantable device 170 to determine the sleep state of the person or subject. In one embodiment, the implantable device 170 could be an electric stimulator device to treat over-active bladder syndrome. The wearable sensor data can measure sleep quality and delivery feedback to the implantable device to correct the over-active bladder syndrome. This information can be used to control implantable devices 170 or external environmental factors in the room where the person or subject is sleeping to improve sleep quality. These environmental factors include the ambient temperature, humidity, noise (e.g., music and environmental noise), and light (e.g., light frequency and amplitude) in the room.

In accordance with some embodiments of the invention, the system can monitor one or more implantable devices 170 or external environmental factors, including ambient temperature, humidity, noise (e.g., music and environmental noise), light (e.g., light frequency and amplitude) in the room and bed firmness, while recording the accelerometer and optionally, EEG data. The accelerometer data and optionally, the EEG data, can be used to detect and identify the sleep states experienced by the person or subject during the night. The system can change one or more environmental factors and observe how it effects the duration of the various sleep states. For example, during deep sleep or rem, the smart phone or hub 130 can increase or decrease the ambient temperature and/or bed firmness and observe whether the change in these environmental parameters causes an increase in the duration of deep sleep or rem sleep. In another example, the smart phone or hub 130 can use the accelerometer data and optionally, the EEG data to strategically select background sounds and/or music to be played during high quality sleep states to induce a deep sleep and/or rem sleep as well as induce longer durations of these sleep states. In accordance with some embodiments, music transitions (e.g., changes in genre/beat/amplitude) can be selected to transition a subject out of deep sleep in time for a pre-scheduled wake up time.

In accordance with some embodiments, the system 100 can (a) establish a correlation between parameters descriptive of music and/or sound (e.g., styles, rhythm genre and/or frequency content, etc.) and parameters indicative of deep sleep (e.g., low heart rate and respiration rate, lower core body temperature, less movement, etc.) across a sample or larger group of users, (b) establish a correlation between parameters descriptive of music and/or sound ((e.g., styles, rhythm genre and/or frequency content, etc.) and parameters indicative of light sleep (e.g., faster heart rate and respiration rate, higher core body temperature, more movement, etc.) across a sample or larger group of users, and (c) controlling the sound environment (e.g., the selected styles, rhythm genre and/or frequency content of sound) accordingly to facilitate or induce a sleep state (e.g., go-to-sleep, enter deep sleep, enter lighter sleep, and/or wake-up) as desired based on the goals of a particular subject.

Music and sound can be used to affect the mental state (e.g. increase or decrease arousal) of a person or subject, and thereby influence the physiology (e.g. increased or decreased HR, galvanic skin response or respiration rate). For example, if an individual is trying to meditate, the wearable sensor can monitor the heart rate, respiration and galvanic skin response to determine the mental state (e.g. relaxed or agitated, and trends or changes in state) and select a song/music which has a calming effect. In accordance with some embodiments, if the determined physiological response (e.g., as measured by the heart rate, respiration rate and galvanic skin response) indicates that the selected song is having (or has had in the past) the desired effect, the song/music/sound would continue playing and any subsequent song/music/sound would be selected to have similar characteristics. On the other hand, if the desired response is not achieved, the system would select a different genre/type of song/music/sound. Over time, the algorithm can learn to select the song/music/sounds or type of song/music/sound that is more likely to help the individual meditate and thus become more personalized.

In accordance with some embodiments of the invention, the system can use a flexible or stretchable conformable sensing device 110 in a system for detecting respiratory paralysis of the expiratory muscles that can accompany Asthma or COPD or Sleep Apnea events and pneumonia. The sensing device 110 can use the accelerometer to monitor respiration function to detect respiration rate, respiration rate variability, depth of breaths, respiration as a function of body position, and/or various kinds of respiratory patterns indicative of problems such as asthma, snoring, sleep apnea, etc. which may have an adverse effect in quality of life, quality of sleep, etc. and statistics derived from the same (e.g. short and long term fluctuations, variance). If respiratory paralysis or signs of impending respiratory paralysis are detected, the smart phone or hub 130 can cause a change in the environment, such as increasing the levels of oxygen and/or nitrogen in the room, causing the smart phone or hub 130 to record the event on a video camera or present a program or application to the person that trains the person to breath rhythmically or causes the person to breath rhythmically (e.g., using music or other organized sounds such as simple tones or beats). In accordance with some embodiments, abnormal breathing patterns can be detected by comparing (and testing for deviations of) real time measurements of sleep-relevant metrics (e.g., heart rate, respiration, motion and temperature based measures) against a normal baseline for a particular subject or a sample or larger group of similar subjects, as well as by comparing such real time measurements against a library of data sets known to contain patterns and metrics indicative of certain abnormalities.

In accordance with some embodiments of the invention, the system can be used to automatically modify the environment of Multiple Sclerosis (MS) patients when they experience a temporary worsening of symptoms due to, for example, an increase in core body temperature. The system according to the invention can include a sensing device 110 that can detect core body temperature as well as worsening of MS related symptoms like gait and balance. The accelerometer data can be used to detect a rise in core body temperature and an associated adverse change in gait and/or balance. In accordance with some embodiments of the invention, adverse changes in gait can be detected and measured by measuring temporal (e.g. stride time, stride time variability, peak angular velocity during swing, double support time) and spatial (e.g. stride length, smoothness of flight path, asymmetry, ground clearance) parameters associated during ambulation. Adverse changes in balance can be detected by measuring transition time (e.g. sit-to stand, lying-to-sitting), sway area of the center of mass while standing, frequency of stumbles/falls and posture.

In accordance with some embodiments of the invention, the system can be used to monitor core body temperature, gait and balance to identify one or more relationships between changes in body temperature and a worsening of MS related symptoms like gait and balance. The sensed body temperature, gait and balance can be used to determine when to send a signal to the thermostat to automatically adjust the ambient temperature or lighting in the room, as a way to automatically improve wellbeing of a patient. For example, in diseases like Multiple Sclerosis, there is a strong causal relationship between core body temperature and temporary worsening of disease symptoms. In accordance with some embodiments of the invention, the sensor device can use the accelerometer data to detect a change (e.g., deterioration or variation) in the measures associated with gait and/or balance and use the temperature sensor to detect and measure an increase in body temperature and be programmed to respond to changes in these conditions by sending a signal to the thermostat to reduce the ambient temperature. Over time, the system would learn this relationship for individual user to optimize this response to prevent/minimize future adverse events.

In accordance with some embodiments of the invention, the system can be used to monitor epilepsy and stroke patients to detect seizure events that occur in the nighttime. These nocturnal seizures are often difficult to diagnose and characterize as they are not directly witnessed by a caregiver or clinician. The system can monitor parameters including HR, respiration rate and motion to build mathematical models or profiles that indicate the onset of such seizures. In accordance with some embodiments of the invention, the system can detect a nocturnal seizure from accelerometer data and cause a predefined action to occur such as sending an alert (e.g., an audible alarm, a text message or phone message) to a family member or care giver, initiating a video recording device to capture the event for retrospective medical decision-making or controlling the lighting system to ease the effects of seizure.

In accordance with some embodiments of the invention, the system can be used to assist patients with Parkinson's Disease (PD), multiple sclerosis (MS), Huntington's disease, essential tremor, depression, ADHD, and OCD who may experience difficulty performing common activities of daily living due to their motor symptoms like tremor, bradykinesia (slowness) and dyskinesia (involuntary jerking). Such activities include eating, using a computer (mouse/keyboard) and driving. In accordance with some embodiments of the invention, the system can be used to monitor and measure one or more characteristics (e.g., frequency, amplitude etc.) of the motion due to the PD or MS symptoms and communicate with external systems and devices to introduce changes in their environment. For example, where the system determines that the person is experiencing tremors or jerking of their hands, while driving a motor vehicle, the system can communicate with a controller in the vehicle or the steering system directly to cause damping of the steering wheel. Severe tremors can interfere with the ability of an individual with Parkinson's disease to drive because of the excessive shaking makes it hard to use the steering wheel to control the car. The frequency associated with tremor in patients with Parkinson's disease is typically between 3-8 Hz. In accordance with some embodiments of the invention, a sensor device 110 placed on the affected arm or hand of a person or subject can be used detect the presence or absence and severity of tremor at any given time. Based on the tremor profile, the steering wheel controller in the car can be controlled by the sensor device 110 or the smart hub 120 to adjust itself to reject steering wheel movements in the tremor frequency band.

Another such example is that of a patient with tremor using a computer mouse. Shaking associated with tremor can make it difficult for an individual to use a mouse to operate a computer. In accordance with the invention, a software filter in the computer can be tuned to the frequency of the tremor motion and controlled by the sensor device 110 (or the smart hub 130) to reject high frequency movements; thus making the mouse easier to use.

In certain patient population (e.g. Parkinson's disease), rhythmic cueing and music or sound can have an immediate impact on the physical status of the person or subject. For example, patients with Parkinson's disease often experience gait disturbances (e.g. freezing of gait) where it becomes difficult for an individual to initiate gait or walk with a regular gait profile. Rhythmic beats and certain music or sound patterns have shown to influence gait in patients with PD. A wearable sensing device 110 can be used to detect gait abnormalities in a patient and automatically send a message to the smart hub 130 to play a rhythmic pattern that could alleviate the problem and help the individual improve their gait. See, for example, McIntosh G C, Brown S H, Rice R R, Thaut M H. Rhythmic auditory-motor facilitation of gait patterns in patients with Parkinson's disease. Journal of Neurology, Neurosurgery, and Psychiatry 1997; 62(1):22-26, which is hereby incorporated by reference.

Rhythmic music or sound and temperature have been shown to help promote relaxation and sleep among patients who would otherwise be wakened by involuntary movements. Music therapists have reported various rhythms or musical styles that can help with walking, balance, and movement in PD and MS patients. Control of this music in the environment through direct wireless connection to a wearable device that monitors symptoms, can thus serve as an automated way to control the symptoms of Parkinson's disease.

In accordance with some embodiments of the invention, the system can be used to control actuated prosthetic devices. For example, the sensing device 110 can detect EMG signals adjacent an amputated limb and use that signal to control actuators in the prosthesis. EMG activation patterns can be detected from muscles adjacent to the amputated limb and used to measure and/or determining intent or characterize fatigue/cramping to improve the control of the prosthetic device. In addition, other sensing devices 110 can be used to sense physical parameters from one or more locations on the body to detect, for example, center of mass motion from the waist/chest or arm swing from the wrist or heart rate) and control or adjust the actuators (e.g., softer or harder force, or more support or less support) in the prosthetic device to respond adaptively in order to improve the operation and performance of the prosthetic device.

In accordance with some embodiments of the invention, the sensing devices 100 can be applied to the body in locations away from the amputation site to detect patterns in the motion and physiological signals that could be used to control the functioning of a prosthetic device. For example, accelerometer data can be used to tracking the center of mass motion of a person or subject to detect or measure the person or subject's balance (e.g., balance, unbalanced, or falling) and send signals to the prosthesis to control the prosthetic device to improve balance. In accordance with some embodiments of the invention, the sensing device 110 can also monitor the heart rate. respiration rate and motion of an individual to determine and measure energy expenditure and fatigue level of the person or subject to control the prosthetic device to increase or decrease the power output. For example, if the heart rate and respiration rate are elevated (perhaps because of steep terrain, which can be detected using the GPS on the smartphone 130), the sensing device 110 or smart hub 130 could control the prosthetic device to increase the power output to make it easier for the individual to walk.

In accordance with some embodiments of the invention, the sensing device 110 can be worn on the body and used to monitor heart function and respiration function (sensor data) while the person or subject is exercising on an exercise machine (e.g., a treadmill, an elliptical machine, a stair climbing machine or a rowing machine). During the exercise period, the sensor data can be used to determine a level stress as a function of one or more of these parameters (e.gl, heart rate, heart rate variability, recovery rate, and respiration rate) and the level of stress can be used to change the operation (e.g., speed and/or resistance) of the exercise machine to provide for optimum training.

In accordance with some embodiments of the invention, the sensor data can be transmitted to the exercise machine wherein a data processing system connected to the exercise machine similar to smart hub 130 processes the sensor data to determine a level of stress and a desirable level of speed and/or resistance for the person on the exercise machine for optimal training. The data processing system in the exercise machine can change the speed and/or resistance dynamically as function of the determined level of stress to provide for optimal training. In accordance with some embodiments, the sensor data can be sent to a smart phone or hub 130 that processes the sensor data and determines the optimal training speed and resistance for the person. The smart phone or hub 130 can communicate with exercise machine to change the speed and/or resistance of the machine. For health and safety purposes, where the sensor data indicates that a dangerous health condition exists, the system receiving the sensor data (e.g., the data processing system in the exercise machine or the smart phone or hub 130) can cause the exercise machine to stop to prevent injury to the person using it.

Over time, the data processing system or the smart phone or hub 130 can modify the selected training speed or resistance as function of past workouts. For example, where the person was able to sustain a predefined level (e.g., speed and/or resistance) for a specific duration of time in the past, the system can increase the speed, resistance and/or duration to enhance training. Further, using a combination of the past workout profiles and physiological responses like heart rate, heart rate variability and heart rate recovery in the context of exercise intensity level, the system can determine or measure an indication of whether the person or subject is over training (e.g., and recommend a reduction or change in exercise level or intensity) or under training (e.g., and recommend an increase or change in exercise level or intensity).

In accordance with some embodiments of the invention, the system can be used monitor the operators of motor vehicles including automobiles, trains, boats and airplanes. For example, where the sensor data indicates that the person is entering into or is in a disabled condition (e.g, a stroke or heart attack), the smart phone or hub 130 or the vehicle control system can cause the vehicle to stop moving and/or alert another operator or a central station of the condition. In this embodiment, the smart phone or hub 130 can communicate with vehicle control system to disable or slow the engine, apply the brakes or other remedial action to avoid injury when the operator becomes disabled. Alternatively, the vehicle can include a controller or system that receives the sensor data directly and automatically initiates a control process that disables or slows the engine, applies the brakes or initiates other remedial action to avoid injury when the operator becomes disabled. Similarly, where the sensor data indicates that person is intoxicated or impaired, for example based on erratic or unstable movements, the vehicle can be prevented from starting or otherwise put into gear.

In accordance with some embodiments of the invention, the system can be used to control high performance aircraft and military aircraft based on sensor data indicating an unsafe condition of the pilot. For example, where the sensor data indicates that the pilot (under the current flight trajectory) will be exposed or is being exposed to a high stress conduction (e.g., excessive G forces as a result of an aerial maneuver) that is likely to cause the pilot to black out or become injured, a control system within the aircraft can modify the flight path and or speed of the aircraft to reduce the stress and avoid harm to the pilot and the aircraft.

In accordance with some embodiments, the system according to the invention can be used to monitor the performance of teams of people or subjects (e.g., dog sled teams or horse teams). In this embodiment, each member of the team can wear one or more sensing devices 110 that generate sensor data for each member of the team. The sensor data can be transmitted to a smart phone or hub 130 worn by one or more members of the team or positioned sufficiently near the field of play to receive the sensor data. The smart phone or hub 130 can aggregate the sensor data for each member of the team and determine one or more condition metrics for each member of the team. The coaching staff can use this information for player selection and substitution during an athletic contest. For example, by monitoring heart rate recovery, the system can rank each member of the team to identify those members of the team exhibiting the least level of fatigue at any point during an athletic contest, thereby enabling the coaching staff to determine which players to utilize during the duration.

In accordance with some embodiments of the invention, the system can be used with a head impact detection system, such as the Checklight product (Reebok, Canton, Mass.), which determines a level of head impact and provides an indication of a possible concussion. The system can use a conformable sensing device 110 worn on the chest and produce sensor data that can be used to determine heart rate, heart rate recovery, and respiration. The system can collect and store sensor data prior to head impact and compare the sensor data after the head impact to determine an indication of the severity of the impact and potential injury.

Other embodiments are within the scope and spirit of the invention. For example, due to the nature of software, functions described above can be implemented using software, hardware, firmware, hardwiring, or combinations of any of these. Features implementing functions may also be physically located at various positions, including being distributed such that portions of functions are implemented at different physical locations.

Further, while the description above refers to the invention, the description may include more than one invention.

What is claimed is:

1. A system comprising:
a sensing device having at least one sensor configured to sense at least one condition of a person and transmit sensor data about the at least one sensed condition; and
an implantable device having electrical and/or chemical actuators configured to deliver therapy upon receiving the sensor data from the sensing device.

2. The system according to claim 1 wherein the sensing device includes two or more components mounted on two or more device islands, wherein each device island is connected to at least one other device island by a flexible or stretchable interconnect enabling the sensing device to flex or stretch and conform to an irregular surface.

3. The system according to claim 1 wherein the sensing device includes an accelerometer adapted for sensing motion of the person.

4. The system according to claim 1 wherein the sensing device includes a sensor that measures bio-potentials.

5. The system according to claim 4 wherein the bio-potentials include at least one of EKG, EMG and EEG.

6. The system according to claim 1 wherein the sensing device includes a sensor that measures at least one of salt concentration in sweat, glucose concentration in sweat, and sweat loss rate.

7. The system according to claim 1 wherein the sensing device includes a sensor that measures at least one of blood micronutrient levels and blood oxygen levels.

8. The system according to claim 1 wherein the sensing device includes a sensor that measures at least one of visible radiation exposure, infrared radiation exposure and ultraviolet radiation exposure.

9. The system according to claim 1 wherein the sensing device includes a sensor that measures at least one of strain, skin strain, and skin modulus.

10. The system according to claim 1 wherein the sensing device includes a sensor that measures at least one of temperature and heat transfer.

11. The system according to claim 1 wherein the sensing device includes a sensor that measures blood glucose and the implantable device includes a chemical actuator that is configured to deliver insulin to a blood vessel.

12. The system according to claim 1 wherein the sensing device includes a sensor that measures a heart rate and the implantable device includes an electrical actuator that stimulates the heart muscle.

13. The system according to claim 1 further comprising an external hub in communication with the sensing device and configured to receive sensor data from the sensing device and transmit the received sensor data to the implantable device.

14. The system according to claim 1 further comprising an external hub in communication with the sensing device and configured to receive sensor data from the sensing device, process the received sensor data and transmit the processed sensor data to the implantable device.

15. The system according to claim 1 wherein the implantable device is configured to provide at least one of electrical stimulation, drug delivery, temperature, ultrasound stimulation, photostimulation, thermal stimulation.

16. A system comprising:
a sensing device having at least one sensor configured to sense at least one condition of a person; and
a target device in communication with the sensing device and configured to receive sensor data from the sensing device and change at least one operating parameter of the target device as a function of the sensor data received from the sensing device.

17. The system according to claim 16 wherein the sensing device includes two or more components mounted on two or more device islands, wherein each device island is connected to at least one other device island by a flexible or stretchable interconnect enabling the sensing device to flex or stretch and conform to an irregular surface.

18. The system according to claim 16 wherein the sensing device includes a sensor that measures bio-potentials.

19. The system according to claim 18 wherein the bio-potentials include at least one of EKG, EMG and EEG.

20. The system according to claim 16 wherein the sensing device includes a sensor that measures at least one of salt concentration in sweat, glucose concentration in sweat, and sweat loss rate.

21. The system according to claim 16 wherein the sensing device includes a sensor that measures at least one of blood micronutrient levels and blood oxygen levels.

22. The system according to claim 16 wherein the sensing device includes a sensor that measures at least one of visible radiation exposure, infrared radiation exposure and ultraviolet radiation exposure.

23. The system according to claim 16 wherein the sensing device includes a sensor that measures at least one of strain, skin strain, and skin modulus.

24. The system according to claim 16 wherein the sensing device includes a sensor that measures at least one of temperature and heat transfer.

25. The system according to claim 16 wherein the target device controls at least one of temperature, humidity, illumination or sound of an environment.

26. The system according to claim 25 wherein the target device includes a thermostat having an adjustable set-point temperature and the target device adjusts the set-point temperature as a function of the at least one sensed condition.

27. The system according to claim 16 wherein the sensing device includes a sensor that measures at least one of a heart rate and a respiration rate and transmits at least one of heart rate sensor data and respiration rate sensor data to the target device and the target device includes at least one of a gaming or virtual reality console that presents a virtual world to the person and wherein the at least one of a gaming or virtual reality console receives at least one of the heart rate sensor data and the respiration rate sensor data and changes the presentation of the virtual world as a function of the at least one of the heart rate sensor data and the respiration rate sensor data.

28. The system according to claim 16 further comprising an external hub in communication with the sensing device and configured to receive sensor data from the sensing device and transmit the received sensor data to the target device.

29. The system according to claim 16 further comprising an external hub in communication with the sensing device and configured to receive sensor data from the sensing device, process the received sensor data and transmit the processed sensor data to the target device.
